(19) United States
(12) Patent Application Publication
(10) Pub. No.: US 2008/0215252 A1
(43) Pub. Date: Sep. 4, 2008

(54) Method of Determining Base Sequence of Nucleic Acid and Apparatus Therefor

(76) Inventors: Tomoji Kawai, Osaka (JP); Hiroyuki Tanaka, Osaka (JP)

Correspondence Address: Cheng Law Group, PLLC, 1100 17th Street, N.W., Suite 503, Washington, DC, 20036, US

(21) Appl. No.: 11/996,647

(22) PCT Filed: Jul. 21, 2006
(86) PCT No.: PCT/JP2006/314494

§ 371 Date: Jan. 24, 2008

(30) Foreign Application Priority Data
Jul. 25, 2005 (JP) 2005-214806

(51) Int. Cl. C12Q 1/68

(52) U.S. Cl. 702/20

(57) ABSTRACT

In a preferred embodiment, an exploring needle of a probe 2 is located at the position of each base of a nucleic acid 6, and a tunneling current value is set to a given value measure. When a bias voltage applied to a substrate is changed step by step from −6 V to 4 V, according to the height of an observed image of each base, the electronic state distribution pattern of each base is obtained. The thus obtained electronic state distribution pattern of each base in the nucleic acid as a measurement object is checked against those in a database to find a base species having the highest degree of similarity to each base by pattern matching to identify each base species to determine the base sequence of the nucleic acid.

### TECHNICAL FIELD

The present invention relates to the determination of the base sequence of a nucleic acid (DNA or RNA).

### BACKGROUND ART

As methods of determining the base sequence of nucleic acid such as DNA or RNA, the Maxam-Gilbert method and the Sanger method have been widely used. Among these methods, the Sanger method is a method of determining the base sequence, in which complementary strands of DNA having various chain lengths are replicated by utilizing the reaction between target DNA and a fluorescently-labeled DNA elongation inhibitor, and are then electrophoresed to determine the base sequence of the target DNA. At present, the Sanger method is mainly used to analyze the base sequence of DNA, and various improvements are being made to the Sanger method (see Patent Document 1).

In recent years, a method of detecting mutation (SNPs: Single Nucleotide Polymorphism) in the base sequence of a specific gene using a DNA array has been developed and commercialized. In this method, fluorescently-labeled unknown DNA is hybridized with known DNA immobilized on a substrate to determine the base sequence of the unknown DNA based on the base sequence of the known DNA bonded to the fluorescently-labeled unknown DNA (see Patent Document 2).

There are also proposed other methods for determining the base sequence of a nucleic acid, by optically observing fluorescence resonance energy transfer (FRET) occurring between a fluorescently-labeled DNA polymerase immobilized on a substrate and type-specifically fluorescently-labeled nucleotides during the synthesis of a complementary strand (see Patent Document 3) and by measuring the three-dimensional structure or shape of self-assembled or hybridized DNA using a scanning probe microscope (see Patent Documents 4 and 5).

Patent Document 1: Japanese Patent Application Laid-open No. H5-038299

### Patent Document 2: Published Japanese Translation of PCT Application No. 2003-528626

Patent Document 3: U.S. Pat. No. 6,210,896B1
Patent Document 4: Japanese Patent Application Laid-open No. H9-299087
Patent Document 5: Japanese Patent Application Laid-open No. H10-215899
Patent Document 6: Japanese Patent Application Laid-open No. H10-282040

### Non-patent Document 1: “Protein, Nucleic Acid, and Enzyme”, Vol. 48, No. 5, pp. 614-620 (2003)

### DISCLOSURE OF THE INVENTION

### Problems to be Solved by the Invention

The Sanger method, which is conventionally and widely used, includes the following steps: (1) amplifying target single-stranded DNA; (2) elongating complementary strand DNA in a solution containing an elongation inhibitor; and (3) separating generated DNA by electrophoresis. These steps involve complicated procedures and take a long time. In addition, fluorescently-labeled nucleotides (Sanger's reagent) used in the elongation step are very expensive and significantly obstruct a reduction in analysis costs. In addition, the length of a base sequence which can be analyzed at one time by electrophoresis performed in the final step is limited to about 800 bases. Analysis speed can be improved by performing parallel electrophoresis using a plurality of capillaries as electrophoretic lanes, but under the current technique, it is difficult to achieve significantly-improved analysis speed absolutely necessary to allow the Sanger method to be applied to customized medicine.

The single nucleotide polymorphism analysis method using a DNA array is becoming mainstream in recent years because analysis speed can be easily improved by increasing the degree of integration of DNA probes immobilized on a substrate. However, this method involves problems such as misreading of a sequence resulting from mismatching and measurement errors caused by the existence of target DNA nonspecifically adsorbed to a substrate support. Further, like the Sanger method, this method also requires amplification of target DNA as pretreatment to improve detection sensitivity, and hence increases the possibility of appearance of false positives resulting from the scattering of amplified products as the degree of integration of DNA probes is increased.

The method of determining the base sequence of a nucleic acid by utilizing fluorescence resonance energy transfer is expected to significantly improve analysis speed because it utilizes the ability of an enzyme to replicate DNA at a rate as high as 1000 bases per second and has theoretically no limitations on the length of a DNA strand that can be read at one time. However, this method involves various problems to be solved for practical use: optical systems in current use do not have high enough sensitivity to allow observation of fluorescence at the single-molecule level required for the method, a failure may occur in synthesizing the enzyme, and the enzyme may be deactivated.

The method of determining the base sequence of DNA by observing the three-dimensional structure or shape of the DNA using a scanning probe microscope also involves various problems which make it difficult to put the method to practical use. For example, data obtained by this method cannot be divided according to the base species to obtain data unique to each base species, and therefore, it is necessary to analogically determine the base sequence of DNA based on the structure or shape of the entire nucleic acid. In addition, this method also involves construction of a huge database and development of an algorithm for the analogy of the base sequence of a nucleic acid.

It is therefore an object of the present invention to provide a method and apparatus for determining the base sequence of a nucleic acid whereby the time and cost for treating a target nucleic acid can be reduced by eliminating the need for amplification of the target nucleic acid and whereby each base species constituting the target nucleic acid can be easily identified.

### Means for Solving the Problems

According to the present invention, the base sequence of a nucleic acid sample is determined based on a previously prepared database constructed from physical signals, which are obtained using a microprobe and each of which is unique to each base species constituting a nucleic acid, by the method including the steps of: (A) immobilizing a part of the nucleic acid sample, whose base sequence is to be distinguished, on the surface of a substrate with the part of the nucleic acid sample being straightened; (B) extracting a physical signal unique to each base from each base unit in the part of the nucleic acid immobilized on the surface of the substrate by measuring each base constituting the nucleic acid with the use of the probe under the same conditions for obtaining the physical signals for the database; and (C) checking each physical signal extracted from each base unit against the physical signals in the database to identify each base species.

It is preferable that at least the surface of the substrate, on which the nucleic acid is to be immobilized, has conductivity, and as the physical signal, an electrical response to an electric field applied between the probe and each base is measured using a scanning probe microscope as a measuring device having the probe.

According to a preferred embodiment of the present invention, the electric field is a bias voltage applied between the probe and each base, and the electrical response is a change in the height of the image of each base in response to a change in the bias voltage when feedback control is performed so that a tunneling current flowing between the probe and each base is kept constant. It is to be noted that the height of the image of each base means the height of the probe (i.e., the position of the probe in the Z-direction) when feedback control is performed so that the tunneling current is kept constant.

According to another preferred embodiment, the electric field is a bias voltage applied between the probe and each base, and the electrical response is a change in tunneling current flowing between the probe and each base in response to a change in the bias voltage when the distance between the probe and each base is kept constant, that is, an I-V curve obtained by scanning tunneling spectroscopy (STS).

According to yet another preferred embodiment, the electric field is a bias voltage applied between the probe and each base and containing an alternating component, and the electrical response is a change in capacitance between the probe and each base in response to a change in the bias voltage.

According to the present invention, an apparatus of determining a base sequence is provided with a measuring unit for recognizing each base unit of a nucleic acid immobilized on the surface of a substrate with the use of a microprobe and extracting a physical signal unique to each base recognized; a memory unit for storing a database constructed from physical signals, which are obtained using the probe and each of which is unique to each base species constituting a nucleic acid; and a data processing unit for checking each physical signal obtained from a nucleic acid sample using the probe against the physical signals in the database to identify each base species to determine and output the base sequence of the nucleic acid sample.

It is preferable that the data processing unit includes a display unit for outputting the base sequence determined.

It is preferable that the measuring unit includes a scanning probe microscope which can apply an electric field between the probe and each base and can measure an electrical response to the electric field. In this case, the electrical response is used as the physical signal.

According to a preferred embodiment of the measuring unit, the electric field is a bias voltage applied between the probe and each base, and the electrical response is a change in the height of each base in response to a change in the bias voltage when feedback control is performed so that a tunneling current flowing between the probe and each base is kept constant.

According to another preferred embodiment of the measuring unit, the electric field is a bias voltage applied between the probe and each base, and the electrical response is a change in tunneling current value flowing between the probe and each base in response to a change in the bias voltage when the distance between the probe and each base is kept constant. In this case, the tunneling current includes not only the value of the tunneling current itself but also a value obtained by first- or higher-order differentiation of the tunneling current value with respect to the bias voltage.

According to yet another preferred embodiment of the measuring unit, the electric field is a bias voltage containing an alternating component and applied between the probe and each base, and the electrical response is a change in capacitance between the probe and each base in response to a change in the bias voltage.

### EFFECT OF THE INVENTION

According to the present invention, the physical properties of each base constituting a nucleic acid can be directly measured using a probe, and therefore the above-described problems such as deactivation of enzyme and biological misreading occurring in the replication of the nucleic acid can be eliminated.

Further, the base sequence can be determined using one DNA molecule as a measuring object, and therefore it is not necessary to perform PCR amplification and electrophoresis for separation, that is, it is possible to eliminate complicated procedures. In addition, a significant improvement in analysis speed can be expected.

The method is a nondestructive measuring method, and therefore it is possible to repeatedly measure the same sample. In addition, the method does not use light as a means for analysis, and therefore requires no expensive reagents such as fluorescent materials and modified nucleotides. This makes it possible to significantly reduce analysis costs.

Further, by measuring each base, it is possible to obtain a physical signal, on the basis of which the identification of each base is performed, from each base unit, and therefore a database to be used for comparison can be constructed from characteristic patterns of only four kinds of bases constituting a nucleic acid. This makes it possible to construct a simple database.

### BEST MODES FOR CARRYING OUT THE INVENTION

FIG. 1 schematically shows the structure of an apparatus according to one embodiment of the present invention realized by using a scanning probe microscope. FIG. 1(A) is a block diagram, and FIG. 1(B) is a plan view which shows a substrate having a nucleic acid sample immobilized thereon.

The scanning probe microscope is provided as a measuring unit, with a microprobe 2 having an exploring needle at its tip and a control device 4. A nucleic acid sample 6 is immobilized on the surface of a substrate 8, of which the surface at least has conductivity, and the substrate 8 is placed on a stage in the scanning probe microscope. The control device 4 applies a bias voltage Vs between the probe 2 and the surface of the substrate 8 and detects a tunneling current It flowing from the probe 2 to the nucleic acid sample 6, controls the position of the probe 2, and then extracts a physical signal S unique to each base species from each base unit constituting the nucleic acid sample 6.

Examples of such a scanning probe microscope include various kinds of microscopes such as an atomic force microscope and a scanning near-field optical microscope. Preferred is a scanning tunneling microscope. An example of the physical signal S extracted includes an electrical response of each base constituting the nucleic acid 6 to the bias voltage Vs.

An example of the electrical response includes a change in the height of each base, that is, a change in the height of the probe 2 in response to a change in the bias voltage Vs when the height of the probe 2 is feedback (FB)-controlled so that tunneling current It flowing between the probe 2 and each base is kept constant.

Another example of the electrical response includes a change in tunneling current It flowing between the probe 2 and each base or in a value obtained by first- or higher-order differentiation of the tunneling current It in response to a change in the bias voltage Vs when the distance between the probe 2 and each base is kept constant.

Still another example of the electrical response includes a change in capacitance between the probe 2 and each base in response to a change in the bias voltage Vs when the bias voltage Vs contains an alternating component.

A memory unit 10 stores a database constructed from physical signals, which are obtained by using the probe 2 and the control device 4 and each of which is unique to each base species constituting nucleic acid. In recent years, various efforts to use a nucleic acid molecule such as DNA as a minimal molecular device have been actively made, and as a result, there has been a report that the electric conductivity of such a molecular device widely varies depending on the base sequence of the nucleic acid used. It has been considered that such a difference in electric conductivity results from a difference in oxidation-reduction potential among four kinds of bases (i.e., adenine, guanine, cytosine and thymine) constituting a nucleic acid. Therefore, the database is constructed utilizing such a difference in characteristics among these four bases and is stored in the memory unit 10.

The physical signals S for constructing the database are obtained prior to the measurement of a nucleic acid sample as a measuring object, and are then stored in the memory unit 10. Examples of a reference nucleic acid to be used for constructing the database include single-stranded nucleic acids synthesized using one kind of base, single bases, nucleosides, nucleotides, and the like.

A data processing unit 12 checks each physical signal obtained from the nucleic acid sample 8 using the probe 2 against the physical signals in the database stored in the memory unit 10 to identify each base species to determine and output the base sequence of the nucleic acid sample. The data processing unit 12 is connected to a display device provided as an output unit, and therefore each base species identified is displayed on the display device. In this way, the kind of each base constituting the nucleic acid sample is identified one by one along a base sequence to determine the base sequence of the nucleic acid sample.

The data processing unit 12 and the memory unit 10 can be realized by using either a computer exclusive to the scanning probe microscope or a general-purpose personal computer.

Hereinbelow, a method of immobilizing the nucleic acid 6, the base sequence of which is to be decoded, on the substrate 8 will be described. The type of the substrate 8 is not particularly limited as long as at least the surface of the substrate 8 has conductivity, and examples of such a substrate 8 include metal crystalline substrates and metal-evaporated substrates. Examples of a method of immobilizing a nucleic acid on a substrate include a method in which a solution containing a target nucleic acid is instantaneously sprayed onto a substrate under vacuum to remove a volatile component so that only the nucleic acid is immobilized on the surface of the substrate (see Non-patent Document 1) and a method utilizing the interaction between streptavidin and biotin (see Patent Document 6). In the case of immobilizing only bases on a substrate, a vacuum thermal evaporation method can be used. However, in the case of immobilizing DNA or RNA on a substrate, DNA or RNA is decomposed when it is thermally evaporated under vacuum, and therefore a method such as any one of the above-described methods is used.

### EXAMPLES

Hereinbelow, a description will be made about an example of a method of determining the base sequence of a nucleic acid by using a scanning tunneling microscope as a scanning probe microscope to measure, as a physical signal, the dependence of the height of each base on bias voltage that is one example of the dependence of tunneling current flowing between a microprobe and each base on bias voltage.

In the normal measurement mode of the scanning tunneling microscope, a bias voltage is applied between an exploring needle of a probe and a sample to detect a tunneling current flowing between the exploring needle and the sample, and then the distance between the exploring needle and the sample is feedback-controlled so that the tunneling current is kept constant. The probe 2 has piezoelectric devices driven in X-, Y-, and Z-directions, respectively so that the exploring needle of the probe 2 can be moved in X-, Y-, and Z-directions over the surface of the substrate 8. It is to be noted that the surface of the substrate 8 is defined as an X-Y plane and a direction toward the probe 2 from the surface of the substrate 8 is defined as a Z-direction.

A database is constructed in the following manner. Four TE (Tris-HCl EDTA-Na₂) solutions each containing any one of four kinds of bases (i.e., adenine, guanine, cytosine and thymine) are prepared, and each of the TE solutions is applied onto a Cu (111) substrate to immobilize the base on the substrate by vacuum thermal evaporation. Then, the tunneling current is set to a constant value in the range of 5 pA to 10 pA, and a bias voltage applied to the substrate is changed step by step from −6 V to 4 V to measure the height (waveform height) of an observed image of the base.

FIG. 2 is a graph in which the horizontal axis represents a bias voltage applied and the vertical axis represents the height of an observed image of each base species. As can be seen from the graph shown in FIG. 2, the dependence of the height of an observed pattern on bias voltage is different according to the kind of base. Such a difference in the height of an observed pattern reflects a difference in electronic state distribution of occupied and unoccupied orbitals of π electron system of each base species, and represents oxidation reduction potential unique to each base species. The database is constructed from the thus obtained electronic state distribution patterns of these bases and is stored in the memory unit 10.

Then, the nucleic acid sample immobilized on the substrate 8 is measured.

First, a constant direct bias voltage is applied between the exploring needle of the probe 2 and the substrate 8 of the scanning tunneling microscope in its normal measurement mode to scan the nucleic acid 6 with the probe 2 in the X-Y direction. When the exploring needle of the probe 2 comes close to the nucleic acid 6 so that the distance between the exploring needle and the nucleic acid 6 becomes nm order during the scanning of the nucleic acid 6 in the X-Y direction, a tunneling current flows between the exploring needle of the probe 2 and the nucleic acid 6 due to tunneling effect. The control unit 4 amplifies the tunneling current, and then a z-direction control voltage for driving the Z-direction piezoelectric device of the probe 2 is applied to the Z-direction piezoelectric device to keep the tunneling current constant so that the height of the exploring needle of the probe 2 is controlled. As a result, an image of the nucleic acid 6 is obtained, and therefore the position of each base in the image can be determined.

Then, based on the thus obtained image of the nucleic acid 6, each base whose position has been determined is identified in the following manner. The exploring needle of the probe 2 is located at the position of each base to measure the height of an observed image of each base under the same conditions for obtaining physical signals for constructing the database, that is, under conditions where the tunneling current is set to a constant value in the range of 5 pA to 10 pA and a bias voltage applied to the substrate is changed step by step from −6 V to 4 V to obtain an electric state distribution pattern. The thus obtained electronic state distribution pattern of each base in the nucleic acid as a measurement object is checked against those in the database to find a base species having the highest degree of similarity to each base by pattern matching, and as a result, the kind of each base is identified. In this way, each base species constituting a target nucleic acid is identified one by one along a base sequence to obtain time-series data, and then the base sequence of the target nucleic acid is determined based on the time-series data.

The method of determining the base sequence of a nucleic acid described above determines the base sequence of the nucleic acid based on the dependence of the height of an observed image of each base on bias voltage, but the base sequence of the nucleic acid can be determined also by using a unique spectral pattern such as an I-V curve obtained by scanning tunneling spectroscopy. In the case of using scanning tunneling spectroscopy, a change in tunneling current dependent on a change in bias voltage is obtained as an I-V curve by sweeping the bias voltage applied between the exploring needle of the probe 2 and the nucleic acid 6 while the distance between the exploring needle of the probe 2 and the nucleic acid 6 is kept constant, and then each base species is identified using the I-V curve as a physical signal.

Further, the base sequence of the nucleic acid can be determined also by using a physical signal extracted using, for example, a commercially-available capacitance bridge, such as a difference in the size of a tunnel barrier according to a base species or a difference in capacitance between a microprobe and each base or in the frequency response thereof according to the base species.

### INDUSTRIAL APPLICABILITY

The present invention can be applied to the determination of the base sequence of DNA or RNA.

### BRIEF DESCRIPTION OF THE DRAWINGS

FIG. 1(A) is a block diagram which schematically shows an embodiment according to the present invention realized by using a scanning tunneling microscope, and FIG. 1(B) is a plan view which shows a substrate having a nucleic acid sample immobilized thereon.

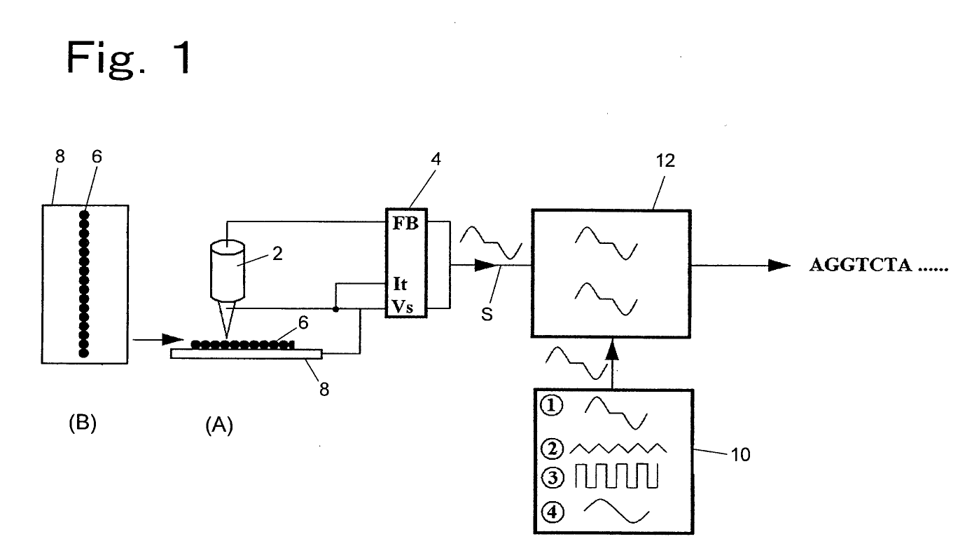

FIG. 2 is a graph which shows an example of a database according to the embodiment of the present invention.

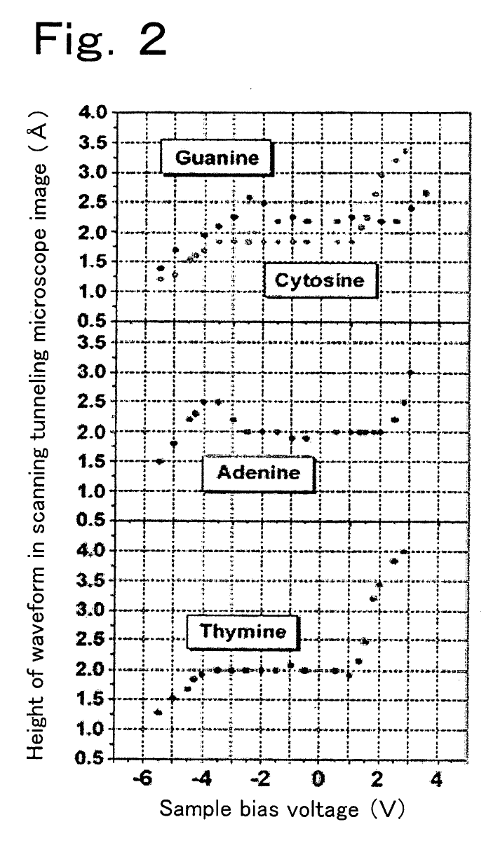

### Explanation of Reference Numerals

2 microprobe
4 control device
6 nucleic acid
8 substrate
10 memory unit
12 data processing unit

1. A method of determining the base sequence of a nucleic acid sample, the method comprising the steps of:
(A) constructing a database containing only electrical responses of four kinds of bases as physical signals, the electrical responses having been obtained as an electrical response of each base unit to a change in bias voltage applied between a microprobe of a scanning probe microscope and a conductive surface of a substrate with reference nucleic acid being immobilized on the conductive surface as a physical signal unique to each base species constituting a nucleic acid;
(B) immobilizing a part of the nucleic acid sample, whose base sequence is to be distinguished, on the conductive surface of the substrate with the part of the nucleic acid sample being straightened, and extracting an electrical response of each base unit to a change in the bias voltage applied between the probe and the substrate as the physical signal unique to each base species constituting the nucleic acid under the same conditions for obtaining the physical signals for the database; and
(C) checking each physical signal extracted from the nucleic acid sample against the physical signals in the database to identify each base species.

2. (canceled)

3. The method of determining the base sequence of a nucleic acid sample according to claim 1, wherein the electrical response is a change in the height of the image of each base in response to a change in the bias voltage when feedback control is performed so that a tunneling current flowing between the probe and each base is kept constant.

4. The method of determining the base sequence of a nucleic acid sample according to claim 1, wherein the electrical response is a change in tunneling current flowing between the probe and each base in response to a change in the bias voltage when the distance between the probe and each base is kept constant.

5. The method of determining the base sequence of a nucleic acid sample according to claim 1, wherein the bias voltage contains an alternating component, and the electrical response is a change in capacitance between the probe and each base in response to a change in the bias voltage.

6. An apparatus for determining a base sequence comprising:
a measuring unit including a scanning probe microscope for recognizing each base unit of a nucleic acid immobilized on the conductive surface of a substrate with the use of a microprobe and extracting an electrical response to a change in a bias voltage applied between the probe and the substrate as a physical signal unique to each base recognized;
a memory unit for storing a database constructed from only the electrical responses as physical signals, which are obtained using the probe and each of which is unique to each of four kinds of base species constituting a nucleic acid; and
a data processing unit for checking each electrical response as a physical signal obtained from a nucleic acid sample using the probe against the electrical responses as physical signals stored in the database to identify each base species to determine and output the base sequence of the nucleic acid sample.

7. The apparatus for determining a base sequence according to claim 6, wherein the data processing unit comprises a display unit for outputting the base sequence determined.

8. (canceled)

9. The apparatus for determining a base sequence according to claim 6, wherein the measuring unit performs feedback control so that a tunneling current flowing between the probe and each base is kept constant to measure as the electrical response, a change in the height of each base in response to a change in the bias voltage.

10. The apparatus for determining a base sequence according to claim 6 wherein the measuring unit keeps the distance between the probe and each base constant to measure as the electrical response, a change in tunneling current flowing between the probe and each base in response to a chance in the bias voltage.

11. The apparatus for determining a base sequence according to claim 6, wherein the bias voltage contains an alternating component, and the electrical response is a change in capacitance between the probe and each base in response to a change in the bias voltage.
